(19) United States
(12) Patent Application Publication
(10) Pub. No.: US 2010/0185035 A1
(43) Pub. Date: Jul. 22, 2010

(54) Nb/Mordenite Transalkylation Catalyst

(75) Inventors: James Butler, League City, TX (US); Olga Khabashesku, Houston, TX (US); Darek Wachowicz, Friendswood, TX (US); Callum Bailey, Humble, TX (US)

Correspondence Address: FINA TECHNOLOGY INC, PO BOX 674412, HOUSTON, TX, 77267-4412, US

(73) Assignee: Fin Technology Inc., Houston, TX (US)

(21) Appl. No.: 12/748,953

(22) Filed: Mar. 29, 2010

Related U.S. Application Data
(63) Continuation-in-part of application No. 12/193,685, filed on Aug. 18, 2008.

(51) Int. Cl. C07C 5/52; B01J 29/076; B01J 29/26

(52) U.S. Cl. 585/475; 502/60; 502/78

(57) ABSTRACT

A niobium-modified mordenite catalyst can be made from water soluble niobium precursors such as niobium oxalate and ammonium niobate(V) oxalate and can be used in toluene disproportionation reactions. Embodiments can provide a toluene conversion of at least 30 wt % of the toluene feed with selectivity to benzene above 40 wt % of the reaction product composition and to xylenes above 40 wt % of the reaction product composition and non-aromatics selectivity of less than 1.0 wt % of the reaction product composition.

### CROSS-REFERENCE TO RELATED APPLICATIONS

The present application is a continuation-in-part of U.S. application Ser. No. 12/193,685 filed on Aug. 18, 2008.

### FIELD

The present invention generally relates to the disproportionation of alkylaromatic feedstreams.

### BACKGROUND

The disproportionation of toluene involves a well known transalkylation reaction in which toluene is converted to benzene and xylene, often referred to as a Toluene Disproportionation Process or TDP, in accordance with the following reaction:

Toluene Disproportionation: Toluene←→Benzene+Xylene (1)

Mordenite is one of a number of molecular sieve catalysts useful in the transalkylation of alkylaromatic compounds. TDP mordenite catalysts generally require a sulfiding procedure to be carried out prior to their use, in order to avoid an initial high percentage of non-aromatics in the product stream. Such non-aromatics can be hard to remove from the product stream because they boil at around the same conditions as benzene.

It is desirable to increase efficiency of the toluene disproportionation process. Longer run times and fewer process shutdowns increase production efficiency and lower associated costs, and this increase in efficiency can be achieved in part by lowering operating temperatures. In view of the above, it would be desirable to have a process of conducting toluene disproportionation with lower production of non-aromatic compounds, with a catalyst that can be operated at lower temperatures.

### SUMMARY

Embodiments of the present invention generally include a niobium-modified molecular sieve catalyst, used in the conversion of hydrocarbons. The niobium deposited on the catalyst support can come from precursors that are water soluble such as chosen from the group consisting of niobium oxalate and ammonium niobate(V) oxalate.

In one embodiment, the molecular sieve is a zeolite. In another embodiment, the zeolite is mordenite.

In one embodiment, the conversion of hydrocarbons consists of a transalkylation reaction, comprising the disproportionation of C₇ to C₁₂ alkylaromatics. In one embodiment, the reaction is a toluene disproportionation reaction (TDP).

In one embodiment, a toluene disproportionation reaction results in a 30 wt % toluene conversion of the toluene feed. In another embodiment, the reaction results in less than 1.0 wt % non-aromatic products of the reaction product stream composition, not considering unreacted toluene.

An alternate embodiment of the present invention is a process for disproportionation of toluene to benzene and xylene that includes passing a toluene/hydrogen feedstock over a niobium-mordenite catalyst at reaction conditions sufficient to provide toluene conversion of at least 30 wt % of the toluene feed. The process can provide non-aromatic selectivity of less than 1.0 wt % of the reaction product stream composition. The niobium content of the catalyst can be between 0.005 wt % to 5.0 wt % by weight niobium metal on the catalyst. The niobium precursor can be chosen from the group consisting of water soluble niobium compounds. The niobium precursor can be chosen from the group consisting of niobium oxalate and ammonium niobate(V) oxalate. The reaction temperature can be between 150° C. and 500° C., optionally between 300° C. and 400° C.

### BRIEF DESCRIPTION OF DRAWINGS

FIG. 1 is a graph of conversion versus temperature for two Nb-Mordenite catalysts.

FIG. 2 is a chart showing selectivity to certain products for a Nb(4 wt %)-Mordenite catalyst.

FIG. 3 is a chart showing toluene conversion and reaction temperature for a Nb(4 wt %)-Mordenite catalyst.

### DETAILED DESCRIPTION

Mordenite is a crystalline aluminosilicate zeolite exhibiting a network of silicon and aluminum atoms interlinked by oxygen atoms within the crystalline structure. For a general description of mordenite catalysts, reference is made to Kirk-Othmer, Encyclopedia of Chemical Technology, 3rd Edition, 1981, under the heading “Molecular Sieves”, Vol. 15, pages 638-643, which is incorporated by reference herein. Mordenite, as found in nature or as synthesized to replicate the naturally occurring zeolite, typically exhibits a relatively low silica-to-alumina mole ratio of about 10 or less. Also known, however, are mordenite catalysts exhibiting substantially lower alumina content. These alumina deficient mordenite catalysts exhibit silica-to-alumina ratios greater than 10, ranging up to about 100, and may be prepared by direct synthesis as disclosed, for example, in U.S. Pat. No. 3,436,174 to Sand or by acid extraction of a more conventionally prepared mordenite as disclosed in U.S. Pat. No. 3,480,539 to Voorhies et al, both of which are incorporated by reference herein. Both those mordenites having silica-to-alumina mole ratio of 10 or less and the aluminum deficient mordenites are known to be useful in the disproportionation of toluene.

Disproportionation of toluene feedstock may be performed at temperatures ranging from 150° C. to 600° C. or above. Optionally the temperature can range from 200° C. to 500° C., or from 250° C. to 450° C. Toluene disproportionation may be at pressures ranging from atmospheric to 1500 psig or above, optionally from 200 psig to 1000 psig, optionally from 400 psig to 800 psig. The liquid hourly space velocities (LHSV) can range from around 1 hr⁻¹ to 10 hr⁻¹, optionally 2 hr⁻¹ to 8 hr⁻¹, and optionally 3 hr⁻¹ to 6 hr⁻¹. The specific catalyst, however, may impose constraints on reaction temperatures in terms of catalyst activity and aging characteristics. In general relatively high temperatures are used when employing the high alumina mordenites (low silica-to-alumina ratios) and somewhat lower temperatures when employing the low alumina mordenites (high silica-to-alumina ratios). Accordingly, where mordenite catalysts exhibiting high silica/alumina ratios have been employed in the transalkylation of alkylaromatics, it has been the practice to operate toward the lower end of the temperature range.

Hydrogen is generally supplied along with toluene to the reaction zone. While the disproportionation reaction (1) does not involve chemical consumption of hydrogen, the use of a hydrogen co-feed is generally considered to prolong the useful life of the catalyst. The amount of hydrogen supplied, which normally is measured in terms of the hydrogen/toluene mole ratio, is generally shown in the prior art to increase as temperature increases. The hydrogen:toluene mole ratio can generally range from 0.05:1 to 5:1, optionally from 0.5:1 to 4:1, optionally from 1:1 to 3:1.

A catalyst comprising a substrate that supports a promoting metal or a combination of metals can be used to catalyze hydrocarbon reactions. The method of preparing the catalyst, pretreatment of the catalyst, and reaction conditions can influence the conversion, selectivity, and yield of the reactions.

The various elements that make up the catalyst can be derived from any suitable source, such as in their elemental form, or in compounds or coordination complexes of an organic or inorganic nature, such as carbonates, oxides, hydroxides, nitrates, acetates, chlorides, phosphates, sulfides and sulfonates. The elements and/or compounds can be prepared by any suitable method, known in the art, for the preparation of such materials.

The term “substrate” as used herein is not meant to indicate that this component is necessarily inactive, while the other metals and/or promoters are the active species. On the contrary, the substrate can be an active part of the catalyst. The term “substrate” would merely imply that the substrate makes up a significant quantity, generally 10% or more by weight, of the entire catalyst. The promoters individually can range from 0.01% to 60% by weight of the catalyst, optionally from 0.01% to 50%. If more than one promoters are combined, they together generally can range from 0.01% up to 70% by weight of the catalyst. The elements of the catalyst composition can be provided from any suitable source, such as in its elemental form, as a salt, as a coordination compound, etc.

In one embodiment, the catalyst can be prepared by combining a substrate with at least one promoter element. Embodiments of a substrate can be a molecular sieve, from either natural or synthetic sources. Zeolites can be an effective substrate, can be commercially available, and are well known in the art. Alternate molecular sieves also contemplated are zeolite-like materials such as for example crystalline silicoaluminophosphates (SAPO) and the aluminophosphates (ALPO).

The present invention is not limited by the method of catalyst preparation, and all suitable methods should be considered to fall within the scope herein. Particularly effective techniques are those utilized for the preparation of solid catalysts wherein a molecular sieve is used as a substrate and one or more promoter elements are added. Conventional methods include co-precipitation from an aqueous, an organic, or a combination solution-dispersion, impregnation, dry mixing, wet mixing or the like, alone or in various combinations. In general, any method can be used which provides compositions of matter containing the prescribed components in effective amounts. According to an embodiment the substrate is charged with promoter via an incipient wetness impregnation. Other impregnation techniques such as by soaking, pore volume impregnation, or percolation can optionally be used. Alternate methods such as ion exchange, wash coat, precipitation, and gel formation can also be used. Various methods and procedures for catalyst preparation are listed in the technical report Manual of Methods and Procedures for Catalyst Characterization by J. Haber, J. H. Block and B. Dolmon, published in the International Union of Pure and Applied Chemistry, Volume 67, Nos 8/9, pp. 1257-1306, 1995, incorporated herein in its entirety.

When slurries, precipitates or the like are prepared, they will generally be dried, usually at a temperature sufficient to volatilize the water or other carrier, such as from 100° C. to 250° C., with or without vacuum. Irrespective of how the components are combined and irrespective of the source of the components, the dried composition can be calcined in the presence of a free oxygen-containing gas, usually at temperatures between about 300° C. and about 900° C. for from 1 to 24 hours. The calcination can be in an oxygen-containing atmosphere, or alternately in a reducing or inert atmosphere.

The addition of a support material to improve the catalyst physical properties is possible within the present invention. Binder material, extrusion aids or other additives can be added to the catalyst composition or the final catalyst composition can be added to a structured material that provides a support structure. For example, the final catalyst composition can include an alumina or aluminate framework as a support. Upon calcination these elements can be altered, such as through oxidation which would increase the relative content of oxygen within the final catalyst structure. The combination of the catalyst of the present invention combined with additional elements such as a binder, extrusion aid, structured material, or other additives, and their respective calcination products, are included within the scope of the invention.

The prepared catalyst can be ground, pressed, sieved, shaped and/or otherwise processed into a form suitable for loading into a reactor. The reactor can be any type known in the art, such as a fixed bed, fluidized bed, or swing bed reactor. Optionally an inert material, such as quartz chips, can be used to support the catalyst bed and to place the catalyst within the bed. Depending on the catalyst, a pretreatment of the catalyst may, or may not, be necessary. For the pretreatment, the reactor can be heated to elevated temperatures, such as 200° C. to 900° C. with an air flow, such as 100 mL/min, and held at these conditions for a length of time, such as 1 to 3 hours. Then, the reactor can be brought to the operating temperature of the reactor, for example 150° C. to 500° C., or optionally down to atmospheric or other desired temperature. The reactor can be kept under an inert purge, such as under a nitrogen or helium purge.

TDP mordenite catalysts generally require a sulfiding procedure to be carried out prior to their use, in order to avoid an initial high percentage of non-aromatics. Such non-aromatics can be hard to remove from the product stream because they can boil at approximately the same conditions as benzene.

Sulfiding consists of the process of depositing sulfur on the catalyst. Sulfiding is known in the art and all suitable sulfiding methods should be considered to fall within the scope herein. A generalized sulfiding procedure involves a sulfur-bearing agent and hydrogen in contact with the catalyst at an elevated temperature. The hydrogen reacts with the sulfur-bearing agent to produce hydrogen sulfide (H₂S), which serves as the sulfiding medium. The H₂S reacts with the metallic catalyst, which gives up an oxygen to form water. The sulfur replaces the oxygen on the catalyst. The process generally follows a schedule of four stages that include: a) placing the catalyst and a sulfur-bearing agent, such as dimethyl sulfide or dimethyl sulfoxide, in a reactor that is purged of air and dehydrated, with or without vacuum, temperature can be in the range of 250° F. to 300° F.; b) hydrogen is introduced with the catalyst and sulfur-bearing agent and the temperature is increased, for example from 40° C. to 230° C.; c) sulfiding occurs in an atmosphere of H₂S, temperature can be in the range of 230° C. to 260° C.; d) sulfiding continues in an atmosphere of H₂S at an elevated temperature, such as in the range of 270° C. to 290° C. A minimum of four hours is typically necessary to complete the sulfiding process. In one example the steps of b), c) and d) each take approximately two hours to complete.

The use of Ni/Mordenite molecular sieve catalysts in toluene disproportionation and heavy aromatic conversion reactions is well known in the art. The present invention provides an improved means of conducting these reactions wherein the nonaromatic selectivity is comparable or lower than the currently used Ni/Mordenite catalyst, even without sulfiding.

In accordance with the present invention, there is provided a novel process for the disproportionation of toluene over a metal promoted molecular sieve catalyst in which a Niobium-modified Mordenite catalyst is used, resulting in low amounts of nonaromatics, and allowing for operation at lower temperatures and longer process run times. Nb/mordenite catalysts can produce an initial low percentage of liquid non-aromatics even without the use of a sulfiding procedure, as has been shown in co-pending U.S. patent application Ser. No. 12/193,685 to Butler et al., which is herein incorporated by reference in its entirety and of which this application is a continuation-in-part.

One embodiment of the present invention is a molecular sieve catalyst containing niobium useful in the conversion of hydrocarbons. In an embodiment the molecular sieve catalyst contains at least 0.005 wt % niobium metal on the catalyst, based on the total weight of the catalyst. In alternate embodiments the molecular sieve catalyst contains at least 0.05 wt % niobium or at least 0.5 wt % niobium. In alternate embodiments the molecular sieve catalyst contains up to 2 wt %, or up to 3 wt %, or optionally up to 5 wt % or more niobium. The molecular sieve catalyst can be of any suitable kind, such as one having a substrate of a zeolite. In an embodiment the substrate can be a mordenite zeolite or a faujasite. Alternate molecular sieves are zeolite-like materials such as crystalline silicoaluminophosphates (SAPO) and the aluminophosphates (ALPO).

The precursor for the niobium can be chosen from among any water soluble compounds that contain niobium. The precursor for the niobium can be chosen from among niobium containing water-soluble precursors, such as the following compounds: niobium oxalate hydrate and ammonium niobate(V) oxalate hydrate, as well as any combinations thereof. Niobium containing water-soluble precursors allow for more evenly distributed and accurate niobium loading. These precursors can also allow for higher niobium loadings, which in turn allow for TDP to be operated at lower temperatures, thus delaying catalyst deactivation, maximizing run time and limiting the need for process shutdowns.

The catalyst can be used in transalkylation reactions, such as the disproportionation of an alkyl benzene or mixtures of alkyl benzenes to produce benzene and polyalkyl benzene. For instance, the invention can be used in the disproportionation of relatively heavy aromatics, such as C₈ to C₁₂ alkyl aromatics. The invention is particularly suitable for the disproportionation of toluene, which can optionally be carried out in the presence of heavier alkylaromatics. In an embodiment when used in a toluene disproportionation reaction process, the present invention can provide a toluene conversion of at least 30 wt % of the toluene feed or in an alternate embodiment a toluene conversion of at least 40 wt % of the toluene feed. In an embodiment when used in a toluene disproportionation reaction process, the present invention can provide a non-aromatic selectivity of less than 1.0 wt % of the reaction product stream composition, not including unreacted toluene feed.

An alternate embodiment of the present invention is a process for disproportionation of toluene to benzene and xylene that includes passing a toluene/hydrogen feedstock over a niobium-mordenite catalyst at reaction conditions sufficient to provide toluene conversion of at least 30 wt % of the toluene feed and provide non-aromatic selectivity of less than 1.0 wt % of the reaction product stream composition. The niobium precursor can be any water-soluble niobium compound, such as niobium oxalate hydrate or ammonium niobate(V) oxalate hydrate or combinations thereof. In an embodiment the niobium content of the catalyst can be between 0.005 wt % to 8.0 wt %, optionally 0.1 wt % to 6.0 wt %, optionally 0.5 wt % to 5.0 wt %. In an embodiment the toluene conversion is at least 40 wt % of the toluene feed. The non-aromatic selectivity can be less than 1.0 wt % of the reaction product composition, optionally less than 0.85 wt %, and optionally less than 0.75 wt %. In an embodiment the selectivity to benzene is at least 30 wt % of the reaction product composition, optionally at least 35 wt %, optionally at least 40 wt %. In an embodiment of the invention the selectivity to xylene is at least 30 wt % of the reaction product composition, optionally at least 35 wt %, optionally at least 40 wt %. In an embodiment the selectivity to heavies is less than 20 wt % of the reaction product composition, optionally less than 15 wt %, optionally less than 10 wt %.

In an embodiment the reaction temperature can range from 150° C.-500° C., optionally from 200° C.-450° C., optionally from 300° C.-400° C. The temperature can be adjusted to maintain a certain toluene conversion level, such as 30 wt % of the toluene feed, or optionally 40 wt %, or more. The hydrogen:toluene molar ratio can be between 0.05:1 to 5:1, optionally from 0.5:1 to 4:1, optionally from 1:1 to 3:1. The reaction pressure can range between atmospheric to 1500 psig or above, optionally from 100 psig to 1000 psig, optionally from 200 psig to 800 psig. The LHSV can be from 1 hr⁻¹ to 10 hr⁻¹, optionally 1 hr⁻¹ to 7 hr⁻¹, and optionally 1 hr⁻¹ to 4 hr⁻¹.

In yet another embodiment of the present invention a process for disproportionation of toluene to benzene and xylene includes passing a toluene/hydrogen feedstock over a niobium-mordenite catalyst with a niobium content of the catalyst of at least 0.05 wt % by weight niobium metal on the catalyst. The reaction conditions are sufficient to provide toluene conversion of at least 30 wt % of the toluene feed and include a reaction temperature between 150° C. and 500° C. and reaction pressure between 200 psig to 800 psig. The non-aromatic selectivity is less than 1.0 wt % of the reaction product composition and the process is capable of such conversion for at least 25 days.

### EXAMPLE

Three Nb/Mordenite catalysts containing 2 wt %, 3 wt %, and 4 wt % of niobium, respectively, were prepared and used in experimental TDP runs. Zeolyst Mordenite Extrudate was used as the base material and was impregnated with niobium using an insipient wetness impregnation technique. The niobium precursor used was ammonium niobate oxalate. For each catalyst, ammonium niobate oxalate hydrate dissolved in 13.5 g of water was deposited on 30 g of mordenite extrudate support. For a 2 wt % loading of niobium metal on the catalyst, 2.537 g of ammonium niobate oxalate hydrate was used. For a 3 wt % loading of niobium metal on the catalyst, 3.806 g of ammonium niobate oxalate hydrate was used. For a 4 wt % loading of niobium metal on the catalyst, 5.075 g of ammonium niobate oxalate hydrate was used. The aqueous solutions were added dropwise to the mordenite base with mixing. The volume of the solution was calculated based on mordenite pore volume per gram of support such that no moisture was present at the bottom of the dish after the impregnation was completed. The support was dried at 120° C. overnight and calcined at 550° C. for 5 hours. The catalyst was not sulfided.

An example of the preparation procedure for obtaining 3 wt % Nb Mordenite by insipient wetness impregnation is now given. Mordenite extrudate (Zeolyst) and ammonium niobate(V) oxalate hydrate (Aldrich), 99.99% trace metals basis C₄H₄NNbO₉.xH₂O and Molecular Weight 302.98 (anhydrous basis), were used for the catalyst preparation. 30 g of Mordenite zeolite was dried in an oven at 110° overnight. Void volume of Mordenite extrudate was determined as 0.45 cc/g, which corresponds to 13.5 cc of solution that is needed for insipient impregnation of 30 g of mordenite extrudate. 3.806 g of ammonium niobate oxalate hydrate was placed in a beaker with 13.5 ml of deionized water. A milky suspension formed after the addition of ammonium niobate oxalate, but the mixture clarified upon standing without mixing overnight and produced a clear solution. (In another experiment a clear solution was obtained by heating the mixture gently without stirring at ˜40° C. for about 20 minutes.) The Nb containing solution was added dropwise with slow mixing to the mordenite placed in the porcelain dish. No solution was left at the bottom of the dish after impregnation; solution was completely absorbed by the mordenite.

The impregnated catalysts were evaluated in a lab scale reactor for disproportionation of toluene to benzene and xylene. The testing conditions are summarized as following:

| | Niobium - Mordenite catalyst |
|---|---|
| | Nb 2 wt %, 3 wt %, 4 wt % niobium metal on the |
| Reactor - down flow | catalyst |
| Feed | Toluene |
| LHSV | 4/hr |
| H2/HC molar ratio | 2:1 |
| Temperature | Adjusted to hold constant conversion |
| RX Inlet Pressure | 600 psig |
| Target conversion | >40 wt % (<60 wt % toluene in effluent) |
| Catalyst volume | 30 mL |

Each new catalyst was loaded into the reactor at the amount of 22 g, which corresponds to 30 cc volume. The reactor was flushed with flowing nitrogen for 15 minutes and pressure checked. The reactor was switched to hydrogen flow at 1 L/min and the pressure increased to 600 psig. The temperature was ramped at 20° C./hr to 360° C. (680° F.), and then the feed was switched to toluene. No sulfiding was done. The temperature was adjusted slowly attempting to maintain about a 40-45 wt % conversion of the toluene feed.

Table 1 shows the activity and temperatures for the three Nb/mordenite catalysts.

TABLE 1
Activity and Temperatures for Nb-Mordenite catalysts.

| Nb 2 wt % Mordenite | | | | | | | | |
|---|---|---|---|---|---|---|---|---|
| Time | | | | | | | | |
| on | | | Nb 3 wt % Mordenite | | | Nb 4 wt % Mordenite | | |
| Stream | Toluene | Temp | TOS | Toluene | Temp | TOS | Toluene | Temp |
| days | conversion % | ° C. | days | conversion % | ° C. | days | conversion % | ° C. |
| 0 | 32.2 | 383 | 3 | 50.9 | 440 | 1 | 41.8 | 385 |
| 1 | 30.6 | 388 | 4 | 38.8 | 415 | 2 | 42.2 | 396 |
| 2 | 32.0 | 388 | 5 | 41.7 | 423 | 5 | 44.7 | 406 |
| 3 | 36.5 | 398 | 6 | 44.2 | 434 | 7 | 41.6 | 407 |
| 12 | 34.4 | 393 | 7 | 45.5 | 439 | 11 | 42.3 | 418 |
| 13 | 32.7 | 393 | 10 | 44.7 | 449 | 12 | 45.9 | 428 |
| 14 | 33.8 | 398 | 11 | 45.3 | 449 | 13 | 46.2 | 436 |
| 17 | 32.8 | 403 | 12 | 45.0 | 452 | 14 | 47.6 | 441 |
| 18 | 35.8 | 412 | 13 | 45.3 | 452 | 15 | 46.9 | 444 |
| 19 | 39.4 | 424 | 14 | 44.4 | 452 | 16 | 49.2 | 449 |
| 20 | 42.9 | 434 | | | | 19 | 47.0 | 449 |
| | | | | | | 20 | 46.3 | 452 |
| | | | | | | 23 | 42.7 | 444 |
| | | | | | | 25 | 43.1 | 454 |

Activity of Nb catalysts increases with the Nb loading as shown in Table 1. The same level of conversion, ˜39 wt % of the toluene feed is achieved at 424° C. for 2 wt % Nb catalyst, at 415° C. for 3 wt % Nb and at less than 407° C. for 4 wt % Nb. Increasing Nb loading level by 1 wt % niobium metal content of the catalyst is seen to lower the reaction temperature by 8° C. or more, while achieving ˜39 wt % initial conversion level.

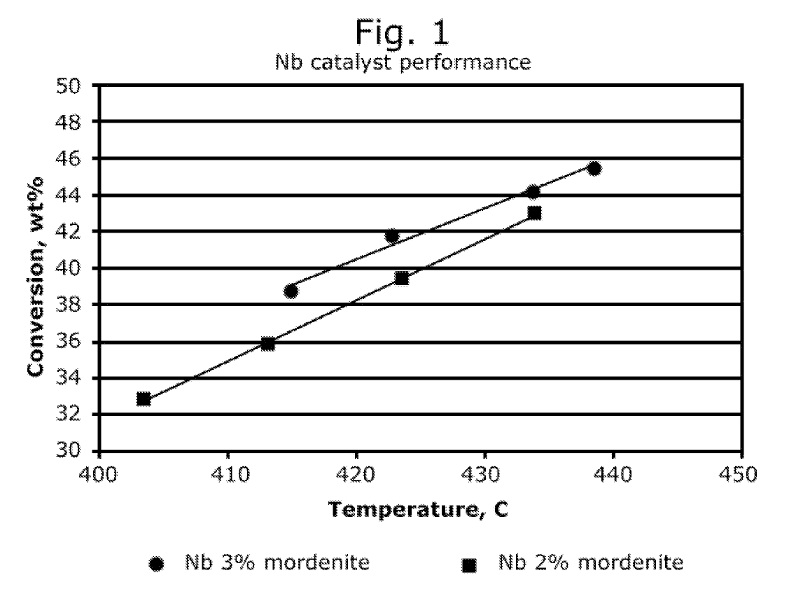

FIG. 1 is a graph showing how conversion, plotted on the y-axis, varies with temperature, plotted on the x-axis. Conversion versus temperature is shown for the 2 wt % and 3 wt % Nb catalysts. As can be seen in FIG. 1, toluene conversion level increases with temperature increase in a linear fashion, and a higher loading of niobium leads to increased catalyst activity. The 3 wt % Nb catalyst performs comparably at lower temperatures than the 2 wt % Nb catalyst.

The above data suggests that higher niobium loadings can allow for TDP reactions to be operated at lower temperatures, which requires less energy and can increase run time, limiting the frequency of process shutdown. The ability to operate at lower temperatures can also allow for operation of the reaction at a higher throughput. Thus, the toluene disproportionation process can be more efficient. Higher loadings of niobium are in part made possible by the use of more water-soluble niobium precursors, like ammonium niobate oxalate hydrate, as such precursors allow for a more even distribution on the mordenite surface and a more accurate niobium loading.

Table 2, below, shows the toluene conversion and selectivity to benzene, xylenes, liquid non-aromatics and C₉+ heavies over Nb(4 wt %)-Mordenite catalyst.

TABLE 2
Toluene conversion and selectivity to products for Nb(4 wt %)-
Mordenite catalyst.

| Time on | Toluene | | | | Liq. | |
|---|---|---|---|---|---|---|
| Stream | Conversion | Benzene | Xylenes | Heavies | Non-ar | Temp |
| Days | wt % | wt % | wt % | wt % | wt % | C. |
| 1 | 41.84 | 44.18 | 46.35 | 7.76 | 0.41 | 385.4 |
| 2 | 42.21 | 44.61 | 45.95 | 8.02 | 0.41 | 395.5 |
| 5 | 44.68 | 44.71 | 46.21 | 7.84 | 0.45 | 405.6 |
| 7 | 41.62 | 44.61 | 46.42 | 7.77 | 0.42 | 407.4 |
| 11 | 42.35 | 44.71 | 45.53 | 8.27 | 0.54 | 417.9 |
| 12 | 45.94 | 43.61 | 45.45 | 9.22 | 0.57 | 428.2 |
| 13 | 46.18 | 44.25 | 44.55 | 9.32 | 0.62 | 435.6 |
| 14 | 47.60 | 44.69 | 44.02 | 9.34 | 0.61 | 440.7 |
| 15 | 46.92 | 43.76 | 44.19 | 9.55 | 0.55 | 443.8 |
| 16 | 49.16 | 42.59 | 44.63 | 10.64 | 0.63 | 448.8 |
| 19 | 46.98 | 43.18 | 44.19 | 9.61 | 0.63 | 448.9 |
| 20 | 46.27 | 43.10 | 44.36 | 9.56 | 0.65 | 451.9 |
| 23 | 42.71 | 43.21 | 45.47 | 8.98 | 0.71 | 443.9 |
| 25 | 43.10 | 42.84 | 44.99 | 9.49 | 0.62 | 454.5 |
| Average | 44.83 | 43.86 | 45.16 | 8.96 | 0.56 | 429.2 |

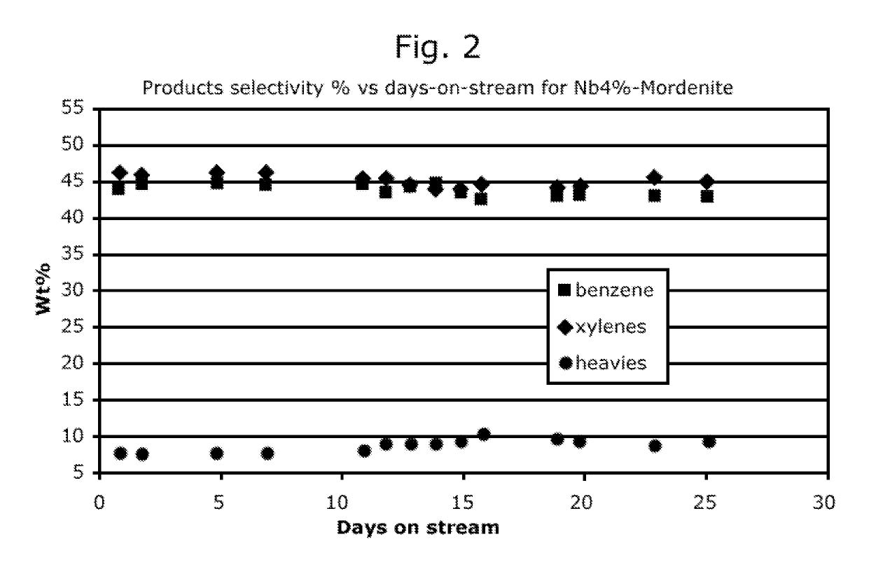

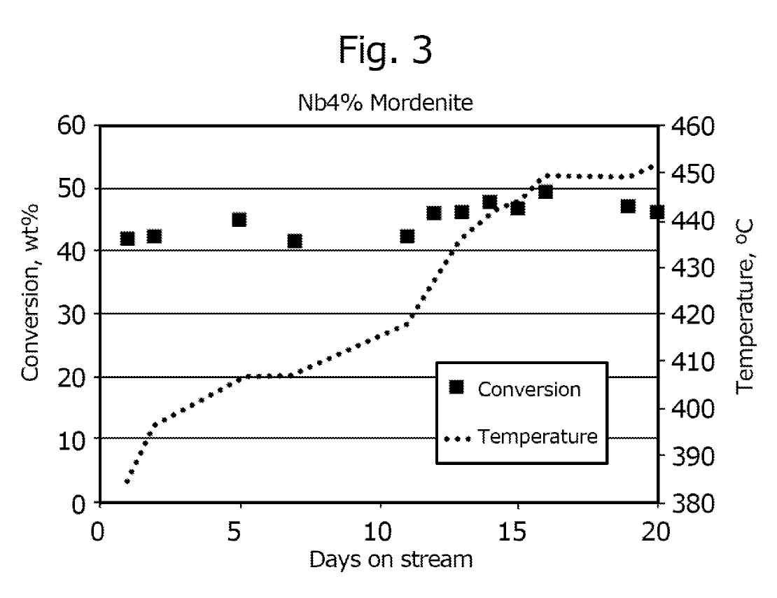

As can be seen from Table 2, the initial production of liquid non-aromatics was low, below 1 wt % of the reaction product composition, and remained below 1 wt % for the first 25 days on stream. The aromatic selectivities were within expected ranges, on average 43.9 wt % of the reaction product composition for benzene, 45.2 wt % of the reaction product composition for xylenes and 9.0 wt % of the reaction product composition for heavies. FIG. 2 displays graphically the selectivity to benzene, xylene, and C₉+ heavies. FIG. 3 displays graphically the toluene conversion and reaction temperature.

The Nb/Mordenite of the present invention achieved high selectivity for benzene (high ratio of Benzene/Xylenes in the effluent) and low production of light ends.

The Nb/Mordenite showed high activity and stability for TDP conversion with low nonaromatic production. In this particular study the activity was stable around 430° C. The quantity of nonaromatic compounds in the product with the Nb/Mordenite catalyst without sulfiding was comparable with previous results from the Ni/Mordenite type TDP catalyst with sulfiding. Elimination of the need for sulfiding can reduce cost and may enable shorter start-up times and achieve on-spec products in a shorter amount of time after start-up.

Various terms are used herein, to the extent a term used is not defined herein, it should be given the broadest definition persons in the pertinent art have given that term as reflected in printed publications and issued patents.

The term “activity” refers to the weight of product produced per weight of the catalyst used in a process per hour of reaction at a standard set of conditions (e.g., grams product/gram catalyst/hr).

The term “conversion” refers to the weight percent of a reactant (e.g. toluene) that undergoes a chemical reaction. For example, X_{Tol}=cony of toluene (wt %)=(Tolᵢₙ−Tolₒᵤₜ)/Tolᵢₙ.

The term “deactivated catalyst” refers to a catalyst that has lost enough catalyst activity to no longer be efficient in a specified process. Such efficiency is determined by individual process parameters. A deactivated catalyst generally requires process shut down in order for a regeneration procedure to be carried out.

The term “molecular sieve” refers to a material having a fixed, open-network structure, usually crystalline, that may be used to separate hydrocarbons or other mixtures by selective occlusion of one or more of the constituents, or may be used as a catalyst in a catalytic conversion process.

The term “niobium content of the catalyst” refers to the content of niobium metal on the catalyst by weight as a percentage of the total catalyst weight. It is the weight of the Nb elemental metal and not the entire weight of any possible Nb containing compound, such as an Nb oxide.

The term “regeneration” refers to a process for renewing catalyst activity and/or making a catalyst reusable after its activity has reached a unacceptable/inefficient level. Examples of such regeneration may include passing steam over a catalyst bed or burning off carbon residue, for example.

The term “selectivity” refers to the weight percentage that a particular product comprises out of the total of all the reaction products. The reaction products do not include unreacted feed. For example the selectivity to benzene would be the wt % of the reaction products that is benzene coming from the toluene that has reacted.

The term “zeolite” refers to a molecular sieve containing a silicate lattice, usually in association with some aluminum, boron, gallium, iron, and/or titanium, for example.

Depending on the context, all references herein to the “invention” may in some cases refer to certain specific embodiments only. In other cases it may refer to subject matter recited in one or more, but not necessarily all, of the claims. While the foregoing is directed to embodiments, versions and examples of the present invention, which are included to enable a person of ordinary skill in the art to make and use the inventions when the information in this patent is combined with available information and technology, the inventions are not limited to only these particular embodiments, versions and examples. Other and further embodiments, versions and examples of the invention may be devised without departing from the basic scope thereof and the scope thereof is determined by the claims that follow.

What is claimed is:

1. A catalyst for the conversion of hydrocarbons comprising:
a molecular sieve catalyst containing niobium, wherein the niobium is deposited on the molecular sieve using a niobium precursor which is chosen from the group consisting of water soluble niobium compounds.

2. The catalyst of claim 1, wherein the water soluble niobium compounds are chosen from the group consisting of niobium oxalate and ammonium niobate(V) oxalate.

3. The catalyst of claim 1, wherein the molecular sieve catalyst is a zeolite.

4. The catalyst of claim 1, wherein the molecular sieve catalyst is a mordenite zeolite.

5. The catalyst of claim 1, wherein the conversion of hydrocarbons is a disproportionation reaction of alkylaromatics.

6. The catalyst of claim 5, wherein the alkylaromatics are C₇ to C₁₂ alkylaromatics.

7. The catalyst of claim 5, wherein the alkylaromatics include toluene.

8. The catalyst of claim 1, wherein the conversion of hydrocarbons is a toluene disproportionation reaction process.

9. The catalyst of claim 8, wherein the catalyst provides a toluene conversion of at least 30 wt % of the toluene feed.

10. The catalyst of claim 8, wherein the catalyst provides a non-aromatic selectivity of less than 1.0 wt % of the reaction product composition.

11. The catalyst of claim 1, wherein the molecular sieve catalyst contains at least 0.005 wt % niobium.

12. The catalyst of claim 1, wherein the molecular sieve catalyst contains at least 0.25 wt % niobium.

13. The catalyst of claim 1, wherein the molecular sieve catalyst contains from 1 wt % to 4 wt % niobium.

14. The catalyst of claim 1, wherein the molecular sieve catalyst contains up to 5 wt % niobium.

15. A process for disproportionation of toluene to benzene and xylene, comprising:
passing a toluene/hydrogen feedstock over a niobium-mordenite zeolite catalyst at reaction conditions sufficient to provide toluene conversion of at least 30 wt % of the toluene feed and provide non-aromatic selectivity of less than 1.0 wt % of the reaction product composition, wherein the niobium is deposited on the molecular sieve using a niobium precursor which is chosen from the group consisting of water soluble niobium compounds.

16. The process of claim 15, wherein the niobium precursor is chosen the group consisting of niobium oxalate and ammonium niobate(V) oxalate.

17. The process of claim 15, wherein the niobium content of the catalyst is between 0.005 wt % to 5.0 wt %.

18. The process of claim 15, wherein the niobium content of the catalyst is at least 0.05 wt %.

19. The process of claim 15, wherein the toluene conversion is at least 40 wt % of the toluene feed.

20. The process of claim 15, wherein the reaction temperature ranges from 150° C.-500° C.

21. The process of claim 15, wherein the reaction temperature ranges from 300° C.-400° C.

22. The process of claim 15, wherein the non-aromatic selectivity is less than 0.5 wt % of the reaction product composition.

23. The process of claim 15, wherein the reaction temperature is adjusted to maintain the toluene conversion level of at least 40 wt % of the toluene feed.

24. The process of claim 15, wherein the hydrogen:toluene molar ratio is between 0.05:1 to 4:1.

25. The process of claim 15, wherein the reaction pressure range is between 200 psig to 800 psig.

26. A process for disproportionation of toluene to benzene and xylene, comprising:
passing a toluene/hydrogen feedstock over a niobium-mordenite catalyst at reaction conditions sufficient to provide toluene conversion of at least 30 wt % of the toluene feed;
the niobium precursor is chosen from the group consisting of water soluble niobium compounds;
the niobium content of the catalyst is between 0.05 wt % and 5.0 wt %;
the reaction temperature is between 150° C. and 500° C.;
the reaction pressure is between 200 psig to 800 psig;
the non-aromatic selectivity is less than 1.0 wt % of the reaction product composition; and
the process is capable of such conversion for at least 25 days.

27. The process of claim 26, wherein the niobium precursor is chosen the group consisting of niobium oxalate and ammonium niobate(V) oxalate.
